(19) United States
(12) Patent Application Publication
(10) Pub. No.: US 2022/0401576 A1
(43) Pub. Date: Dec. 22, 2022

(54) GOLD NANOPARTICLES/NANOSHELLS IMMUNE CONJUGATES FOR ENHANCED IMMUNOTHERAPY AND PHOTOTHERMAL THERAPY FOR HEMATOLOGIC MALIGNANCIES

(71) Applicant: Northwestern University, Evanston, IL (US)

(72) Inventors: Adam Y. Lin, Chicago, IL (US); Leo I. Gordon, Evanston, IL (US)

(21) Appl. No.: 17/773,519

(22) PCT Filed: Nov. 2, 2020
(86) PCT No.: PCT/US2020/058474

§ 371 Date: Apr. 29, 2022

Related U.S. Application Data
(60) Provisional application No. 62/929,570, filed on Nov. 1, 2019.

(51) Int. Cl. A61K 47/69; A61K 41/00; A61K 39/39; B82Y 5/00; A61P 35/04

(52) U.S. Cl. CPC A61K 47/6929; A61K 41/0052; A61K 39/39; B82Y 5/00; A61P 35/04; A61K 2039/55561

(57) ABSTRACT

Disclosed herein are CpG conjugated nanoparticles for immunotherapy and photothermal therapy. The composition comprises class B CpG conjugated nanoparticles and/or a class C CpG conjugated nanoparticles where the class B CpG conjugated nanoparticles comprises a nanoparticle core and a class B CpG conjugated thereto and the class C CpG conjugated nanoparticles comprises a nanoparticle core and a class C CpG conjugated thereto.

### CROSS-REFERENCE TO RELATED APPLICATIONS

The present application claims priority to U.S. Provisional Patent Application No. 62/929,570 that was filed Nov. 1, 2019, the entire contents of which are hereby incorporated by reference.

### BACKGROUND OF THE INVENTION

About 80,000 non-Hodgkin's lymphoma (NHL) cases are diagnosed every year in the United States, with an estimated 20,000 deaths in 2019. One third of patients with diffuse large B cell lymphoma (DLBCL), the most common type of aggressive B cell NHL, do not respond to standard chemo-immunotherapy. Patients with high risk features have a 5-year overall survival rate of only 33%. For those who are fit enough to undergo salvage therapy including adoptive cellular therapies, the long term disease free survival is still only about 40%. Therefore, the design and implementation of innovative approaches to improve these outcomes represent an important unmet need.

### BRIEF SUMMARY OF THE INVENTION

Disclosed herein are immune conjugate nanoparticles for immunotherapy and photothermal therapy and methods of making the same. One aspect the invention provides for a composition comprises CpG conjugated nanoparticles. The composition comprises class B CpG conjugated nanoparticles and a class C CpG conjugated nanoparticles where the class B CpG conjugated nanoparticles comprises a first nanoparticle core and a class B CpG conjugated thereto and wherein the class C CpG conjugated nanoparticles comprises a second nanoparticle core and a class C CpG conjugated thereto. In some embodiments, the class B CpG conjugated nanoparticle comprises a first spacer interposed between the class B CpG and the first nanoparticle and/or the class C CpG conjugated nanoparticle comprises a second spacer interposed between the class C CpG and the second nanoparticle.

Another aspect of the invention provides for a method for the inhibition of growth, proliferation, or killing of a cell. The method comprises contacting the cell with the class B CpG conjugated nanoparticle and/or the class C CpG conjugated nanoparticle according to claim 1 under conditions sufficient for inhibiting the growth, proliferation, or killing of the cell. In some embodiments, contacting the cell with the composition leads to apoptosis.

Another aspect of the invention provides for a method for the increased expression of B cell markers, immune modulatory markers, and maturation markers. The method comprises contacting a cell with the class B CpG conjugated nanoparticle and/or the class C CpG conjugated nanoparticle according to claim 1 under conditions sufficient for increasing expression of B cell markers, immune modulatory markers, and maturation markers of the cell. In some embodiments, expression of CD19, CD20, CD40, CD47, CD80, CD86, OX40, or any combination thereof is increased.

Another aspect of the invention provides for the increased expression of cytokines. The method comprises contacting a cell with a class B CpG conjugated nanoparticle and/or a class C CpG conjugated nanoparticle according to claim 1 under conditions sufficient for increasing expression of cytokines of the cell. In some embodiments, the increased expression is of IL-6 or TNF.

In some embodiments, the methods involved contacting a hematologic cancer cell with the class B CpG conjugated nanoparticle and/or a class C CpG conjugated nanoparticle. In particular embodiments, the cell is a lymphoma cell.

In some embodiments, the methods involved contacting an immune cell with the class B CpG conjugated nanoparticle and/or a class C CpG conjugated nanoparticle. In particular embodiments, the cell is an antigen presenting cell or a lymphocyte.

Another aspect of the invention provides for a method for the treatment of a subject in need of a treatment for a cancer. The method comprises administering a therapeutically effective amount of the class B CpG conjugated nanoparticle and/or the class C CpG conjugated nanoparticle or a pharmaceutical composition thereof, the pharmaceutically composition further comprising a pharmaceutically acceptable excipient, carrier, or diluent. In some embodiments, the cancer is a hematological cancer, including, without limitation, lymphoma. In some embodiments, the method further comprises co-administering an immunomodulatory therapy.

These and other aspects of the invention will be described in further detail below.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1E. (FIG. 1A) Schematic of tmCpG NP design. CpG sequences were modified with triethylene glycol (TEG) spacer, polyT sequence, and thiol group that allows for self-assembly on gold nanoparticles. (FIG. 1B) Illustration of the use of triethylene glycol modified CpG nanoparticles (tmCpG NPs) to induce immunogenic cell death via apoptosis. (FIG. 1C) Illustration of the use of triethylene glycol modified CpG nanoparticles (tmCpG NPs) to activate and mature dendritic cells. (FIG. 1D) Illustrates strong in situ vaccination by harnessing the immunogenic properties of photothermal therapy (PTT) using hollow gold nanoshells (HGNs) exposed to near infrared (NIR) light. (FIG. 1E) Absorbance spectra of tmCpGs to 15 nm NPs, including class B murine CpG 1826 (left) and human CpG 7909 (middle), as well as class C CpG 2395 (right)

FIGS. 2A-2C. (FIG. 2A) The duplex nature of class C CpGs, tmCpG 2395 NPs (right) caused aggregates to form post centrifugation (lighter color), while class B CpGs are linear and thus tmCpG 1826 NPs (left) and tmCpG 7909 NPs (middle) suspend well after centrifugation steps. (FIG. 2B) Class C tmCpG 2395 NPs are able to be resuspended into PBS after sonication. (FIG. 2C) Aggregates form when tmCpG 2395 NPs were washed without temperate control. However, if the particles are collected with a temperature controlled microcentrifuge (20° C.), they form a fluidic oil pellet similar to the class B tmCpG NPs and are easily resuspended by vortexing the solution.

FIGS. 3A-3D. (FIG. 3A) Using viability assays, class B tmCpG 1826 NPs had a significant reduction in viability of murine lymphoma cells (A20) compared with free class B CpG at various concentration. (FIG. 3B) At a CpG concentration of 2.5 μg/ml, human class B tmCpG 7909 NPs caused increased cell death of SUDHL4 (GC DLBCL), RC (high-grade lymphoma), and Ramos (Burkitt lymphoma) cells compared with free CpG 7909 conditions. (FIG. 3C) Class C tmCpG 2395 NPs also reduced the viability of murine lymphoma cell line (A20) compared with free class C CpGs as well as (FIG. 3D) human lymphoma cells (SUDHL4, RC, and Ramos). Viability assays were performed at 72 h. *P<0.05; **P<0.01.

FIGS. 4A-4D. Viability assay of human (FIG. 4A) RC, high-grade B lymphoma, (FIG. 4B) JeKo-1, TP53 deficient large cell variant mantle cell lymphoma (MCL), (FIG. 4C) Mino, MCL with TP53 mutation, and (FIG. 4D) REC-1 TP53 proficient MCL cell after treatment with class B (left column) or class C (right column) CpG or tmCpG NPs after 3 days (solid fill) or 5 days (patterned fill). * P<0.05; ** P<0.01.

FIGS. 5A-5D. (FIG. 5A) AnnexinV-PI apoptosis assay of mouse lymphomaA20 cells treated with media only, free mouse classBCpG1826 (2.5 μg/ml), or tmCpG1826NPs (5 nM) after 24 h of treatment. (FIG. 5B) Percent of cells that are in early apoptosis (Annexin V+PI−) or late apoptosis (Annexin V+PI+) within each treatment group. The nanoparticle treatment group induced more early and late apoptosis signatures than free CpG treatment. IL-6 secretion from A20 cells after treatment with (FIG. 5C) media only, class B CpG 1826 (1.25 μg/ml), or tmCpG 1826 NPs (2.5 nM) or (FIG. 5D) class C CpG 2395 (1.25 μg/ml), or tmCpG 2395 NPs (2.5 nM). *P<0.05; **P<0.01.

FIGS. 6A-6F. Viability of murine bone marrow dendritic cells (JAWSII) treated with Free CpG or tmCpG NPs of (FIG. 6A) class B (1826) and (FIG. 6D) class C (2395). (FIG. 6B) IL-6 and (FIG. 6C) TNF secretion from dendritic cells after treatment with class B CpG 1826 or tmCpG 1826 NPs. (FIG. 6E) IL-6 and (FIG. 6F) TNF secretion from dendritic cells after treatment with class C CpG 2395 or tmCpG 2395 NPs. *P<0.05; **P<0.01.

FIGS. 7A-7B. Surface markers changes on murine lymphoma A20 cells after 72 h of treatment with media (filled gray) or (FIG. 7A) class B or (FIG. 7B) class C free CpGs (1.25 μg/ml) (dotted line) or tmCpG NPs (2.5 nM) (solid line). Surface markers included B cell markers CD19 and CD20, immune modulatory markers PDL1, OX40, CD47, maturation markers CD80, CD86, and CD40.

FIGS. 8A-8D. Using a dual tumor model (A20 cells in BALB/c mice), PBS, class B or class C CpG, or class B or class C tmCpG NP were injected in the larger tumor for 3 doses. The spider plots of (FIG. 8A) treated side or (FIG. 8B) untreated side tumor growth after treatment with PBS (left), class B CpG 1826 (middle), or class B tmCpG 1826 NP (right) (n=15 each). In addition, shown are the spider plots of (FIG. 8C) treated or (FIG. 8D) untreated tumor growth after treatment with PBS (left), class C CpG 2395 (middle), or class C tmCpG 2395 NP (right) (n=4 each).

FIGS. 9A-9B. (FIG. 9A) Treatment with B CpG or BNP or BNP+CNP had better direct tumor suppression compared with C CpG or CNP or B+C CpG. (FIG. 9B) BNP+CNP treatment group had significantly better abscopal effect compared with all other conditions which are not different than PBS treatment group.

FIG. 10. Total tumor volume (treated tumor+untreated tumor) over time. The BNP+CNP group had significantly lower tumor volumes compared with all other conditions.

FIGS. 11A-11C. BNP+CNP had improved event free survival in compared with all other groups for any tumor volume greater than 2000 mm³ (FIG. 11A), total tumor volume greater than 2000 mm³ (FIG. 11B), and total tumor volume greater than 3000 mm³ (FIG. 11C).

### DETAILED DESCRIPTION OF THE INVENTION

The disclosed technology may be used for the preparation of immune conjugate nanoparticles for immunotherapy and photothermal therapy. The presently disclosed nanoparticles (NPs) may be used to prepare cancer vaccines, harvest leukemic phase cancers and reinfuse with vaccine or immunotherapy within a close loop system, infusion treatment for lymphoma treatment, or combination immunotherapy, with for example other immunotherapies such as checkpoint inhibitors and CAR T cells, and provide an effective treatment option for these clinically difficult aggressive lymphomas, and the like.

### CpG Conjugated Nanoparticles

The immune conjugate NPs comprise oligodeoxynucleotides (ODNs) comprising unmethylated cytosine-phosphate-guanine (CpG) motifs. CpG ODNs, or CpGs, are single stranded DNA sequences that comprise a phosphodiester link between two consecutive C and G nucleotides. CpGs are typically 50 nucleotides or less. Suitably the CpG may be between 10 and 50 nucleotides, 15 and 45 nucleotides, or 20 and 40 nucleotides. When the CpG motifs are unmethylated, the CpG ODNs and are recognized as immunostimulants or a pathogen-associated molecule pattern (PAMP) due to their abundance in microbial genomes but relative scarcity in vertebrate genomes. CpGs are recognized by pattern recognition receptors (PRR), such as Toll-like Receptor 9 (TLR9).

CpGs may be categorized in one of several different classes. For example, the CpG may be a class A, B, C, P, or S CpG. CpG ODNs possess a partially or completely phosphorothioated (PS) backbone, as opposed to the natural phosphodiester (PO) backbone found in genomic bacterial DNA. The classes of stimulatory CpG ODNs have been identified based on structural characteristics and activity on human peripheral blood mononuclear cells (PBMCs), in particular B cells, and plasmacytoid dendritic cells (pDCs). Class A CpGs or CpG-A ODNs are characterized by a phosphodiester central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. Class B or CpG-B ODNs contain a full PS backbone with one or more CpG dinucleotides. They strongly activate B cells and TLR9-dependent NF-κB signaling but weakly stimulate IFN-α secretion. Class B or CpG-B ODNs combine features of both classes A and B. Class C CpG ODNs contain a complete PS backbone and a CpG-containing palindromic motif. C-Class CpG ODNs induce strong IFN-α production from pDC as well as B cell stimulation. In some embodiments, the nanoparticle or compositions described herein comprise one or both of class B and class C CpGs.

In certain embodiments, compositions comprising both class B and class C CpGs may be prepared. As demonstrated in the examples, compositions comprising both class B and class C CpGs show the strongest abscopal effect that is superior to compositions comprising only one of class B or class C CpGs. The ratio of class B to class C CpGs may be appropriately selected for the desired activity. In some embodiments, the ratio of class B to class C CpGs in the nanoparticles and compositions described herein may be from 10:1 to 1:10, including without limitation 8:1 to 1:8, 6:1 to 1:6, 4:1 to 1:4, 2:1 to 1:2, or approximately 1:1.

Efforts to harness the innate immune system have been successful in cancer therapy, yet the results of checkpoint inhibition in DLBCL have been disappointing and CAR-T therapy still leaves >60% of patients without many options. Synthetic oligodeoxynucleotides containing unmethylated cytosine-phosphate-guanine (CpG) motifs may be used to enhance immune mediated effects. CpGs bind to toll-like receptor 9 (TLR9), which when activated, causes dendritic cell maturation and formation of memory B cells, and induces pro-inflammatory cytokine secretion. CpGs induce tumor specific T cell development, and are especially relevant for B cell lymphomas, which typically express TLR9. CpGs lead to G1-phase arrest and autocrine secretion of interferons and induce apoptosis via the Fas ligand pathway. Therefore, CpGs not only have immune stimulatory effects, but also can lead directly to lymphoma cell death. In addition, CpGs can directly inhibit the immunosuppressive functions of myeloid derived suppressor cells (MDSCs) and result in differentiation into macrophages with antitumor activity. However, the clinical utility of CpGs is limited by the inability to efficiently deliver CPGs directly to lymphoma sites and immune cells and also by the rapid degradation of CPGs. Therefore, the use of NPs for CpG delivery may enhance the efficacy of this approach.

The disclosed technology advantageously allows for the use of NPs as a carrier template for CpG motifs. NPs protect the CpGs from degradation allowing systemic delivery. NPs may also traffic CpG ODN's to their desired target. CpGs may target receptors, such as the toll-like receptor 9 (TLR9) located in the endosomes. NPs, such as gold nanoparticles, naturally accumulate in the endosomes trafficking the CpGs to its target, leading to a stronger immune stimulatory effect. Moreover, NPs may be formulated to absorb light, allowing for photothermal therapy (PTT) that generates a strong in situ vaccination effect. In addition, magnetically responsive hollow nanoshells can be used for MRI contrast agents and cell separation devices.

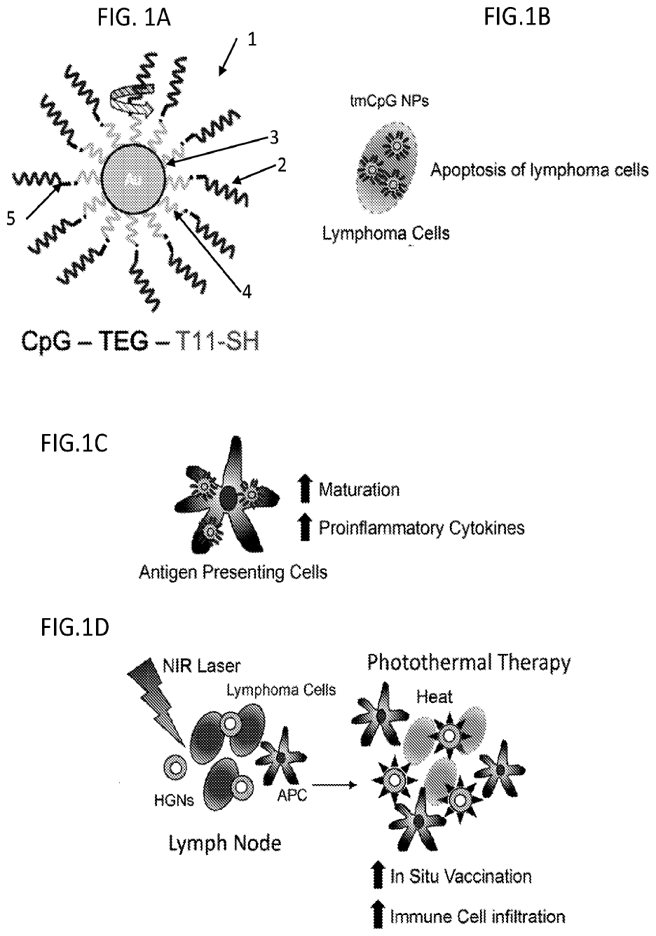

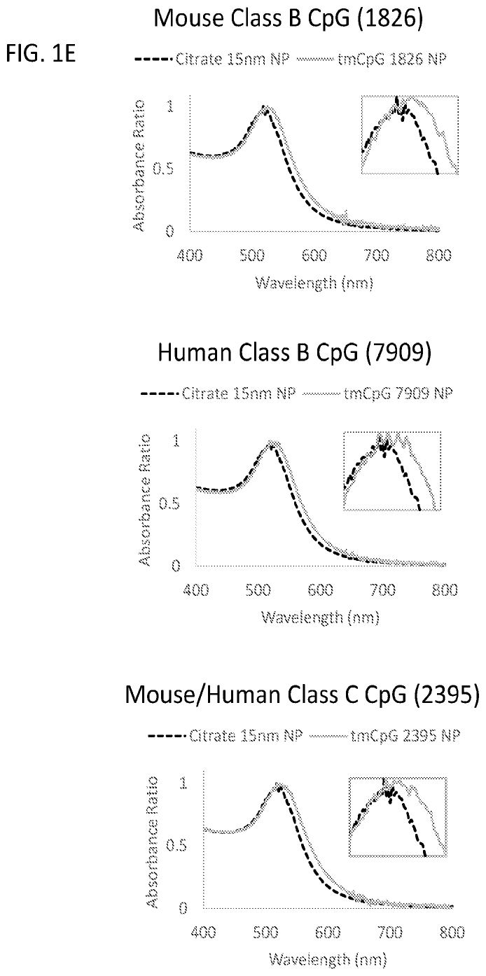

FIG. 1A illustrates an exemplary CpG conjugated nanoparticle 1. The CpG conjugated nanoparticle comprises a CpG 2 conjugated to a NP core 3. Interposed between the CpG and the NP core may be a spacer. In some embodiments, the spacer comprises an oligonucleotide 4 and an oligoethylene glycol 5. The CpG conjugated to the NP core may be any of the CpGs described herein.

The NP core may be substantially uniform throughout or a nanoshell. The NP core may be composed from different materials, including metallic materials, semiconducting materials, oxide materials, photothermal materials, magnetic materials, polymeric, liposomal, and the like. The NP may be between 10 nm and 100 nm in diameter, including without limitation between 10 nm and 80 nm, 10 nm, and 50 nm, or 10 nm and 30 nm. As demonstrated in the Examples, 15 nm diameter core tmCpGNPs were more stable and effective at causing lymphoma cell death than those having larger NP cores.

In some embodiments, the NP core comprises gold. Gold nanoparticles (AuNPs) are suitable nanocarriers for CpG delivery. First, AuNPs are inert, non-toxic, and easily functionalized with thiol-modified synthetic DNA, forming a tight self-assembled monolayer that protects them from degradation. The simplicity of the synthesis and design is critical for scale up in order to have clinical relevance. Second, on the cellular level, AuNPs accumulate in endosomes, which is where the target receptor TLR9 resides. Lastly, systemically delivered AuNPs typically accumulate in the lymphoid organs, which are traditionally viewed as a drawback when trying to deliver chemotherapy, but are important for cancer therapy to generate systemic immunity. Within the spleen, the majority of nanoparticles collect in B cells. Therefore, AuNPs are excellent carriers to passively target malignant B cells. In addition, AuNPs were able to be collected at the tumor site within 24 h after intravenous delivery.

In some embodiments, the CpG conjugated nanoparticle comprises an oligoethylene glycol spacer that provides for rotational or conformational flexibility. In some embodiments, the oligoethylene glycol comprises 2, 3, 4, 5, 6, 7, 8, 9, or 10 ethylene glycols. In particular embodiments, the oligoethylene glycol is a triethylene glycol such as used in the Examples. Triethylene glycol modified CpG sequence (tmCpG) to allow improved rotation and binding to TLR9. These tmCpG NPs induce macrophages to secrete higher amounts of pro-inflammatory cytokines, such as tumor necrosis factor-α (TNFa), interleukin-6 (IL-6), and granulocyte-colony stimulating factor (G-CSF) compared with free CpGs or even nonmodified CpGs directly conjugated on NPs. This stimulation of the macrophages is sequence specific as control GpC sequences did not lead to pro-inflammatory cytokine secretion.

In some embodiments, the CpG conjugated nanoparticle comprises an oligonucleotide spacer. The oligonucleotide spacer elevates the CpG away from the NP core, allowing enough spacing in-between the CpGs to that the CpGs are capable of binding to a receptor. The oligonucleotide spaces may also result in improved stability. The oligonucleotide spacer may be composed of 20 nucleotides or fewer, including without limitation 2, 3, 4, 5, 6, 7, 8, 9, 10, 11, 12, 13, 14, 15, 15, 16, 17, 18, 19, or 20 nucleotides. Suitably, the oligonucleotide spacer is a polyT sequence, such as the 11-mer used in the Examples.

Lymphoma responds differently to class B and class C CpG ODNs. As demonstrated in the Examples, combinations of class B and class C CpGs demonstrated a synergistic effect that is superior to either class B or class C CpGs along. Class B CpGs have a linear structure, while class C CpGs are palindromic and have a duplex secondary structure. Both classes of CpG bind to TLR9 but class B CpGs only stimulate B cells while class C CpGs act on both B cells and dendritic cells. Class C CpGs may be used to activate dendritic cells to boost immunotherapy efficacy and class B CpGs may have a direct cytotoxic effect against hematological malignancies, such as lymphoma.

In some embodiments, compositions described herein comprise two different CpG conjugated NPs, the first CpG conjugated NP comprising a class B CpG conjugated to a NP core and the second CpG conjugated NP comprising a class C CpG conjugated to a NP core. In some embodiments, the ratio of first CpG conjugated NP to second CpG conjugated NP may be from 10:1 to 1:10, including without limitation 8:1 to 1:8, 6:1 to 1:6, 4:1 to 1:4, 2:1 to 1:2, or approximately 1:1. Although each of the first and second NPs may comprises only class B CpGs and only class C CpGs, class B and class C CpGs may be conjugated to the same NP core.

### Pharmaceutical Compositions

Pharmaceutical compositions may be formed from the NPs described herein. The compounds utilized in the methods disclosed herein may be formulated as pharmaceutical compositions that include: (a) a therapeutically effective amount of one or more NPs as described herein; and (b) one or more pharmaceutically acceptable carriers, excipients, or diluents. The NPs utilized in the methods disclosed herein may be formulated as a pharmaceutical composition for delivery via any suitable route. For example, the pharmaceutical composition may be administered as an injectable formulation via intravenous, intramuscular, or subcutaneous.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable. Such pharmaceutical compositions contain an effective amount of a disclosed NPs, which effective amount is related to the daily dose of the compound to be administered. Each dosage unit may contain the daily dose of a given compound or each dosage unit may contain a fraction of the daily dose, such as one-half or one-third of the dose. The amount of NPs to be contained in each dosage unit can depend, in part, on the identity of the particular NP, or combinations of NPs, chosen for the therapy and other factors, such as the indication for which it is given. The compounds for use according to the methods of disclosed herein may be administered as a single NP or a combination of NPs. For example, a NP may be administered as a single NP or in combination with another NPs that promote anti-cancer activity or that has a different pharmacological activity.

### Method for the Treatment of a Subject in Need of a Treatment for a Cancer

Methods for treating cancer, particularly hematological malignancies, in a subject with the use of the NPs described herein are provided. Suitably the method for treating the subject comprises administering to the subject an effective amount of a NPs or a pharmaceutical composition comprising the effective amount of the NPs.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with the NPs disclosed herein. For example, a “subject in need of treatment” may include a subject in need of treatment for a cancer.

In some embodiments, the cancer is a hematological malignancy or a hematological cancer. A hematological cancer is a neoplastic disease of the hematopoietic and lymphoid tissues. Examples of hematological cancers include lymphomas that may affect lymphocytes, such as B Cells and T Cells. In some embodiments, the cancer is a lymphoma, including Non-Hodgkin's lymphoma and Hodgkin's lymphoma. Exemplary Non-Hodgkin's lymphomas include B Cell lymphomas, such as diffuse large B-cell lymphomas (DLBCL), T Cell lymphomas, Burkitt's lymphoma, follicular lymphoma, mantel cell lymphoma, primary mediastinal B cell lymphoma, and small lymphocytic lymphoma, lymphoplasmacytic lymphoma. Exemplary Hodgkin's lymphomas include lymphocyte-depleted Hodgkin's disease, lymphocyte-rich Hodgkin's disease, mixed cellularity Hodgkin's lymphoma, nodular lymphocyte-predominant Hodgkin's disease, and nodular sclerosis Hodgkin's lymphoma

As used herein the term “effective amount” refers to the amount or dose of the compound, upon single or multiple dose administration to the subject, which provides the desired effect in the subject under diagnosis or treatment. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Compositions can be formulated in a unit dosage form. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

### Methods for the Inhibition of Growth, Proliferation, or Killing of a Cell

Another aspect of the invention includes methods for the inhibition of growth, proliferation, or killing of a cell. The method may comprise contacting a cell with any of the NPs described herein under conditions sufficient for inhibiting the growth, proliferation, or killing of the cell. “Conditions sufficient for inhibiting the growth, proliferation, or killing” of the cell means that the growth or proliferation of the cell is inhibited by a statistically significant amount or cell viability is reduced by a statistically significant amount. In some embodiments, the statistically significant amount is a difference relative to control of at least 5%, but in some cases at least 10%, 20%, 30%, 40%, 50%, or more. The methods described herein may be performed in vitro, ex vivo, or in vivo.

As illustrated in FIG. 1B, contacting cells with the NPs described herein may result in apoptosis of the cell. The cell may be a cancer cell. In some embodiments, the cancer cell is a cell associated with a hematologic cancer. Suitably the cancer cell is a lymphoma cancer cell associated with any of the lymphomas described herein.

As demonstrated in the examples, the NPs described herein significantly reduced the viability of a number of different lymphoma cells, including murine lymphoma (A20), DLBCL (SUDHL4), high-grade lymphoma (RC), Burkitt lymphoma (Ramos), mantle cell lymphoma (MCL).

### Methods of Increasing Expression of Cytokines

Another aspect of the invention includes methods for increasing expression of cytokines. The method may comprise contacting a cell with any of the NPs described herein under conditions sufficient for increasing expression of cytokines. “Conditions sufficient for increasing expression of cytokines” of the cell means that expression of at least one cytokine is increased by a statistically significant amount. In some embodiments, the statistically significant amount is a difference relative to control of at least 5%, but in some cases at least 10%, 20%, 30%, 40%, 50%, or more. The methods described herein may be performed in vitro, ex vivo, or in vivo.

Cytokines are category of small proteins, typically on the order of 5-20 kDa, that are important in cell signaling. Cytokines include, for example, chemokines, interferons, interleukins, lymphokines, and tumor necrosis factors. Cytokines are produced by a variety of cell types including immune cells such as B cell, T cells, macrophages, and mast cells. They act through cell surface receptors and modulate humoral and cell-based immune responses and regulate maturation. As illustrated in FIG. 1C, contacting cells with the NPs described herein may result in the increased expression of cytokines. In some embodiments, the cell is an antigen presenting cell (APC), dendritic cell, or a lymphocyte, such as a B cell lymphocyte.

As demonstrated in the examples, the NPs described herein significantly increase the expression of a number of different cytokines, including IL-6 and TNF-α when bone marrow-derived dendritic cells (BMDC) are contacted with the NPs described herein.

### Methods for Increased Expression of B Cell Markers, Immune Modulatory Markers, or Maturation Markers

Another aspect of the invention includes methods for increasing expression of B cell markers, immune modulatory markers, or maturation markers. The method may comprise contacting a cell with any of the NPs described herein under sufficient for increasing expression of B cell markers, immune modulatory markers, and maturation markers of the cell. “Conditions sufficient for increasing expression of B cell markers, immune modulatory markers, or maturation markers” of the cell means that expression of at least one such marker is increased by a statistically significant amount. In some embodiments, the statistically significant amount is a difference relative to control of at least 5%, but in some cases at least 10%, 20%, 30%, 40%, 50%, or more. The methods described herein may be performed in vitro, ex vivo, or in vivo.

The B cell markers, immune modulatory markers, and maturation markers may include cluster of differentiation (CD) markers. CD markers provide a method for identifying or investigating cell surface molecules that may act as receptors or ligand in a signally pathway. In some embodiments, the CD with increased expression is CD19, CD20, CD40, CD47, CD80, CD86, or OX40. As illustrated in FIG. 1C, contacting cells with the NPs described herein may result in the increased expression of the aforementioned markers, resulting in altered behavior of the cell.

As demonstrated in the examples, the NPs described herein significantly increase the expression of a number of different B cell, immune modulatory and maturation markers, including CD19, CD20, CD40, CD47, CD80, CD86, and OX40 when lymphoma cells are contacted with the NPs described herein.

### Combination Therapies

The NPs described herein may be used in a combination therapy with one or more additional therapeutic modalities. In some embodiments, the co-administered modality takes advantages of one or more resultant properties that are a consequence of administration of the NPs described herein, including increased expression of cytokines or increased expression of B cell markers, immune modulatory markers, and maturation markers. As a result, combination therapies including the administration of the NPs described herein with one or more immunomodulatory therapies, such as checkpoint inhibitor therapy, antibody therapy, CAR T cell therapy, or radiation therapy, e.g., photothermal therapy, may be employed. The co-administered therapeutic modality may be contemporaneously administered with the NPs described herein. In other embodiments, the co-administered therapeutic modality may be administered before or after the administrations of the NPs described herein.

Photothermal therapy (PTT) with HGNs can generate a strong vaccination effect in melanoma. Hollow gold nanoshells are small gold structures that can be modified to absorb light and generate heat, which is utilized for photothermal therapy (PTT). PTT increases blood flow in tumors, induces cytotoxicity, and disrupts tumor vasculature, resulting a in situ vaccination effect (FIG. 1D). PTT stimulates a cascade of pro-inflammatory cytokines including IL-6, TNF-α, C-CSF, GMCSF, and CCL2 that can not only promote the expansion of both pro-inflammatory (CD4+ and CD8+) cells but also increases suppressive immune cells. Thus PTT using HGNs in lymphoma will also generate a strong in situ vaccination effect.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Synthesis of Stable Class B and Class C CpGs Conjugated Gold Nanoparticles

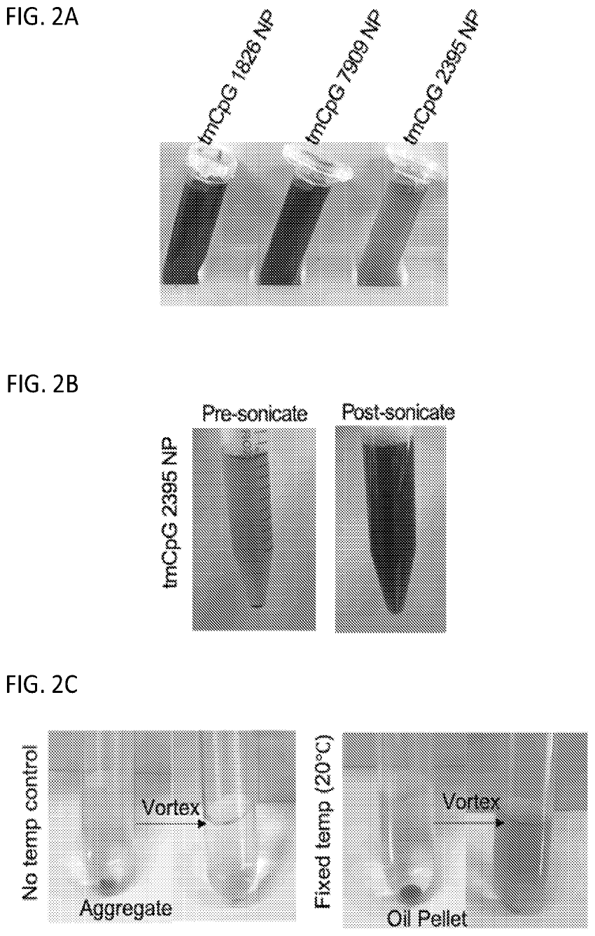

Modified class B (sequence 1826 for mice, and 7909 for human) and class C (sequence 2395 for both mice and humans) CpGs were conjugated onto citrate stabilized 15 nm AuNPs (FIG. 1A). The tmCpG NP peak of the absorbance spectra increased by 3 nm (522 nm to 525 nm) compared with citrate NPs, suggesting successful surface modification (FIG. 1E). Unlike class B tmCpG NPs, class C tmCpG 2395 NPs formed aggregates during centrifugation and washing steps (FIG. 2A). These aggregates did not re-suspend with vortexing. Sonication can resuspend the particles but over time the particles re-aggregated (FIG. 2B). Washing and collection of the tmCpG 2395 NPs using a temperature-controlled centrifuge (20° C.) prevented particles from forming aggregates and resulted in the formation of a fluidic oil pellet similar to class B tmCpG NPs (FIG. 2C). In addition, both class B and class C tmCpG NPs did not have any visible aggregation after incubation with cells and media. Media allow formation of protein corona around the nanoparticles which further provides stability of the construct.

For 60 nM of tmCpG 7909 NP, 42.9±4.4 μg/ml of DNA was conjugated on the gold surface. Thus, 1 nM of tmCpG 7909 NP contains 0.72 μg/ml of DNA, which is equivalent to 0.48 μg/ml of CpG 7909. Similarly, for 60 nM of tmCpG 2395 NP, there was 51.4±2.0 μg/ml of DNA measured. So, 1 nM of tmCpG 2395 NP contains 0.86 μg/ml of DNA, which is equivalent to 0.55 μg/ml of CpG 2395. Therefore, further studies used 1 nM of tmCpG NPs as equivalent to 0.5 μg/ml of CpGs. This matches previously published data showing that about 76 tmCpGs strands were conjugated on a 15 nm gold particle, which equate to 0.537 μg/ml of CpG for 1 nM of the tmCpG NPs.

The tmCpG NP Platform Induced Increased Lymphoma Cell Death Compared with Free CpGs

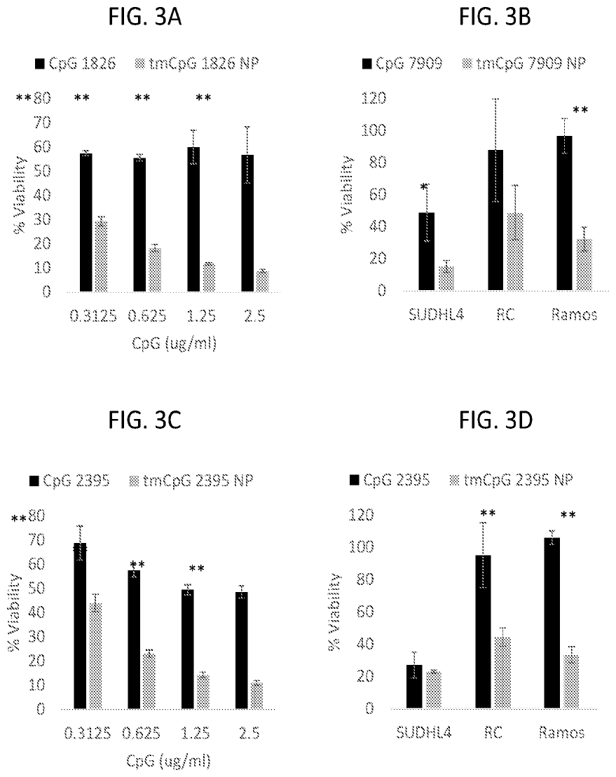

Murine lymphoma (A20) cells were treated with media, free mouse class B CpG 1826 or tmCpG 1826 NPs, and free class C CpG 2395 or tmCpG 2395 NPs at various concentrations for 72 h. 72 h time point showed the most cell death with compared with 24 and 48 h. Mouse class B tmCpG 1826 NPs reduced viability of A20 cells as seen in MTS assays compared with free CpG 1826 at all concentrations (FIG. 3A). At 2.5 μg/ml CpG 1826 or 5 nM of the tmCpG 1826 NPs, the viability of A20 cells was 56.7% versus 8.7%, respectively (P<0.01). Similarly, class C tmCpG 2395 NPs also caused significantly reduced viability of the A20 cells when compared with free CpG 2395. (FIG. 3C).

Furthermore, as shown in FIG. 3B, human class B tmCpG 7909 NPs significantly reduced viability of DLBCL (SUDHL4) and Burkitt lymphoma (Ramos) cells compared with free human CpG 7909 (P<0.05 and P<0.01, respectively). In a high grade B cell lymphoma (RC) cell line, the class B tmCpG 7909 NPs had a viability difference of almost 40% compared with free CpG treated groups (48% vs 87%; P=0.08) (FIG. 3B). Conversely, class C tmCpG 2395 NPs were significantly better at killing RC and Ramos cells than free CpGs (P<0.01 respectively) (FIG. 3, D). In SUDHL4 cells, both free class C CpG 2395 and tmCpG 2395 NPs significantly reduced cell viability (27% and 23%), with no observed statistical significance between the two classes.

Longer Treatment Duration with tmCpG NPs Leads to Increased Lymphoma Cell Death in Human Lymphoma Cell Lines

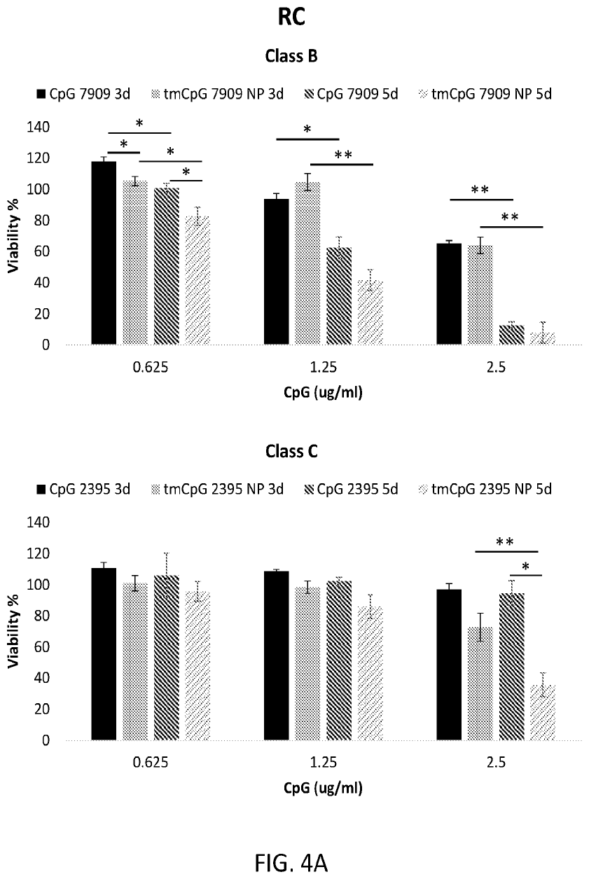

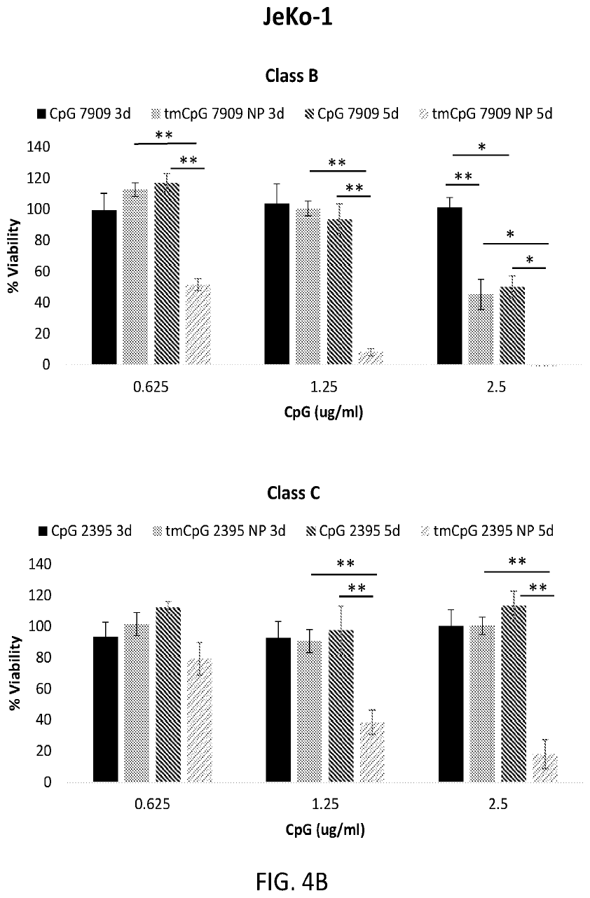

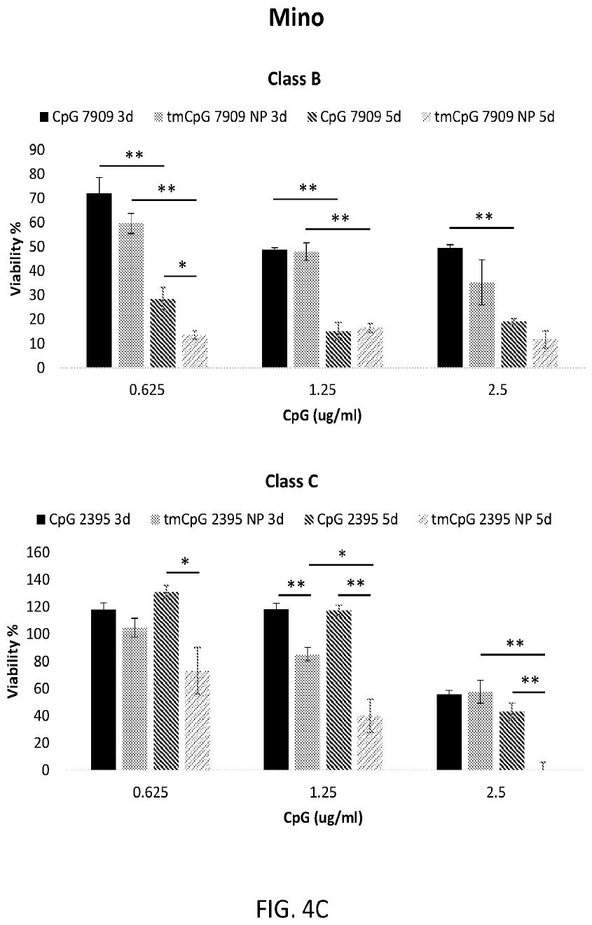

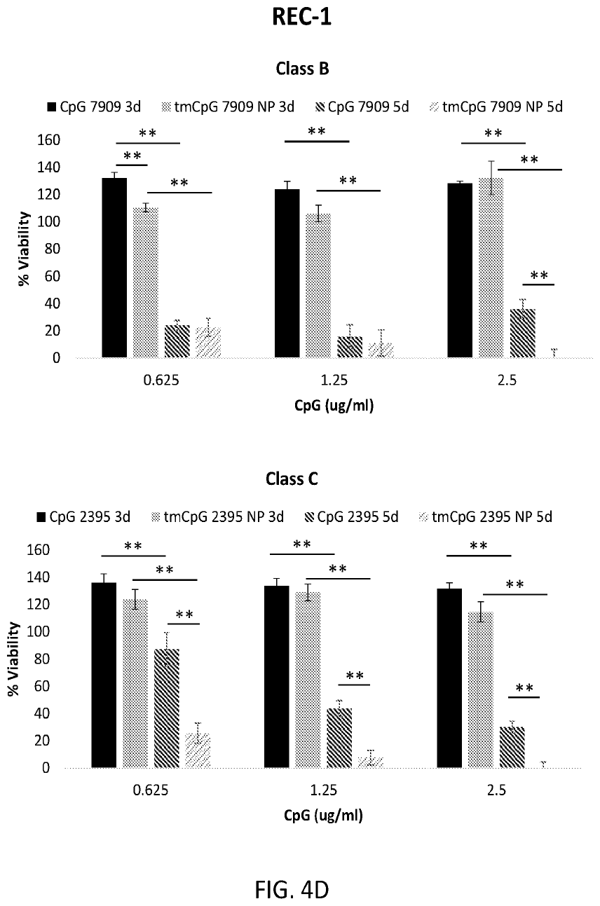

CpG stimulation of lymphoma cells from patients leads to apoptosis despite initial proliferation over time. Significant lymphoma cell death was noted after 4-7 days of treatment. Similarly, treatment of human high-grade lymphoma line and mantle cell lymphoma (MCL) cell lines with tmCpGNPs of either class B (7909) or class C (2395) caused significant reduction of viability after treating for 5 days when compared to 3 days (FIG. 4A-4D). For RC cells, MYC and BCL2 rearranged, 5 days of treatment of class B CpGs resulted in significant reduction in viability for both CpG (12.8% at 2.5 μg/ml) and tmCpG NP (7.97% at 2.5 μg/ml) when compared with treatment after 3 days (CpG 65.2% and tmCpG NP 62% at 2.5 μg/ml). Using the same concentration of 2.5 μg/ml, only 5-day treatment of class C tmCpG NPs showed significant viability reduction of RC cells (3.5%) compared with 3-day treatment of CpG (96.8%), tmCpG NP (72.8%), or even 5-day treatment of CpG (94.5%).

Similar patterns were seen in human MCL cells. JeKo-1, TP53-deficient MCL, which correlates to more aggressive disease, had a dramatic reduction of viability when treatment with class B tmCpG 7909 NPs (0%) and class C tmCpG 2395 NP (18%) after 5 days compared with 3-day treatment (45.3% and 100%, respectively) at 2.5 μg/ml of CpG. Even at lower concentrations, treatment for 5 days had lower viability in all concentrations. Furthermore, using Mino, TP53-mutated MCL, and REC-1, TP53-proficient MCL, cell lines, 5-day treatment with tmCpG NPs for either class B or class C had significant reduction in viability. For all three MCL cell lines, class B CpGs were more cytotoxic than class C CpGs. Also, these results are clinically important for MCL since higher levels of TLR9, which the CpGs target, correlate to worse outcomes.

The 15 nm Diameter Core tmCpG NPs were More Stable and Effective at Causing Lymphoma Cell Death than 30 nm or 50 nm Gold Cores

Lin et al showed that 15 nm tmCpG NPs induced the highest cytokine release from macrophages compared with 30 nm or 80 nm nm NPs.15 Here, class C tmCpG 2395 NPs were synthesized on 15, 30 nm, or 50 nm AuNPs. The 15 nm tmCpG NPs were the most stable during synthesis with the least amount of aggregation. Similarly, 15 nm tmCpG 2395 NPs had improved killing of A20, SUDHL4, and RC cells compared with larger nanoparticle cores of 30 nm or 50 nm, standardized by surface area to estimate similar CpG dosing.

The tmCpG NPs Kill Lymphoma Cells Via Apoptosis

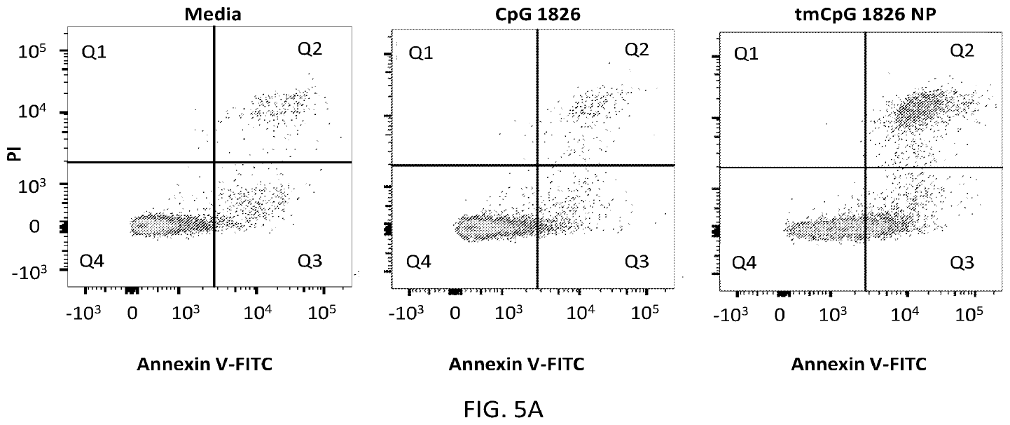

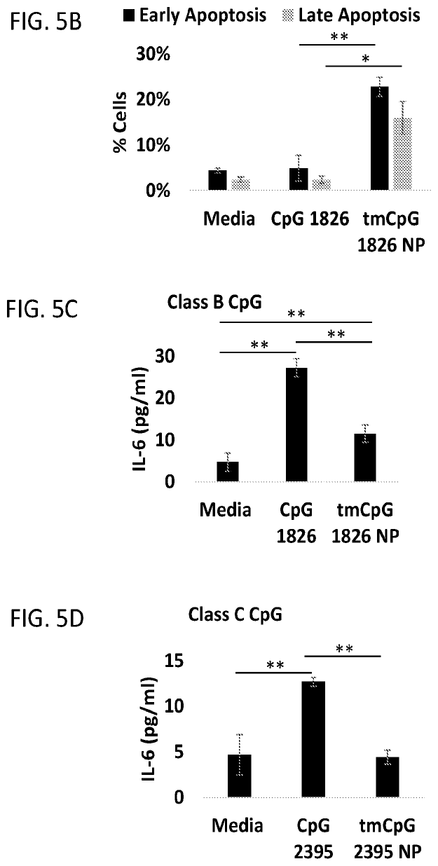

TmCpG 1826 NPs induced lymphoma cell death by apoptosis as early as 24 h after treatment (FIG. 5A). The tmCpG 1826 NP treated A20 cells had significantly more early apoptotic cells (annexin V+PI−) than CpG 1826 treated cells (23% vs 5%). Moreover, the particle treated A20 cells also had more cells in the late apoptosis phase (annexin V+PI+) compared with free CpG treated cells (16% vs 2.4%) (FIG. 5B). Treatment with class B CpG 1826 (1.25 μg/ml) led to higher interleukin-6 (IL-6) (27.3 pg/ml) secretion from A20 cells compared with tmCpG 1826 NP (11.5) or media only (4.7). Similarly, class C CpG 2395 treatment caused higher IL-6 secretion (12.7 pg/ml) compared with tmCpG 2395 NP (4.4) or media only (4.6) (FIG. 5B).

Neither Class B and C tmCpG NPs Altered Dendritic Cell Viability but Class C tmCpG NPs Caused Increased Cytokine Release

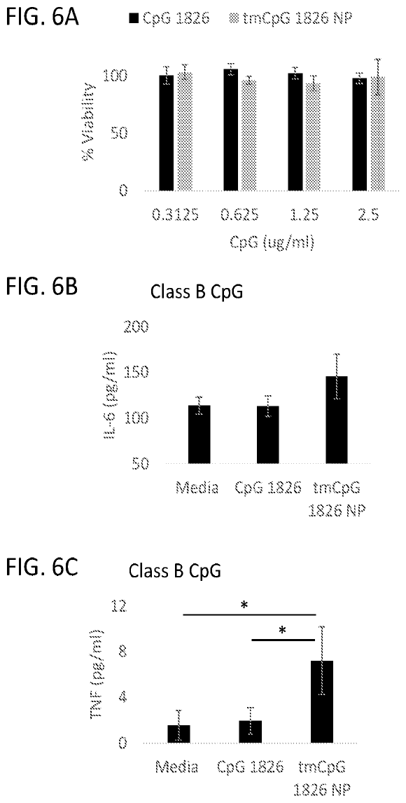

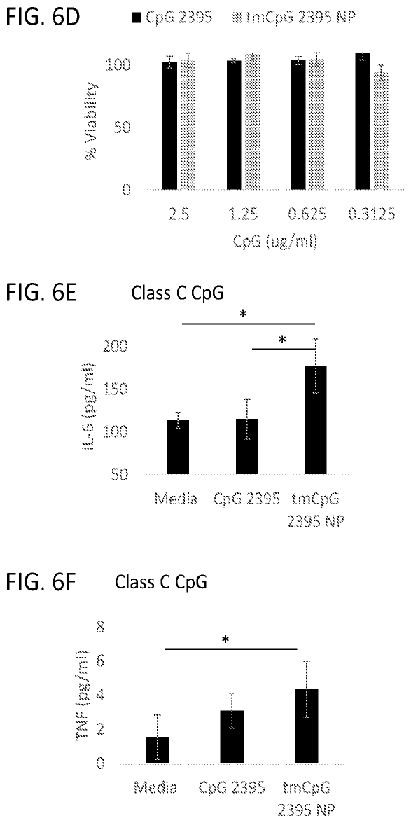

The viability of murine bone marrow-derived dendritic cells (BMDC) was not altered when treated with class B 1826 (FIG. 6A) or class C 2395 (FIG. 6D) CpGs or tmCpG NPs. Mouse class B tmCpG 1826 NPs trended toward an increase in IL-6 (145 pg/ml) from BMDCs compared with free class B CpG1826 (113.1 pg/ml) or media control (113.7 pg/ml) (FIG. 6B), while tmCpG 1826 NPs caused a significant increase in TNFa secretion (7.2 pg/ml) compared with CpG (1.9 pg/ml) and media control (1.5 pg/ml) (FIG. 6C). Class C tmCpG 2395 showed a significant increase in IL-6 secretion (177.5 pg/ml) from JAWSII cells compared with class C CpG (115 pg/ml; P=0.02) (FIG. 6E) and a significant increase of TNFa (4.4 pg/ml) compared with media control (1.5 pg/ml) (FIG. 6F).

### TmCpG NPs Treated Lymphoma Cells Caused Increased Expression of B Cell, Immune Modulatory and Maturation Markers

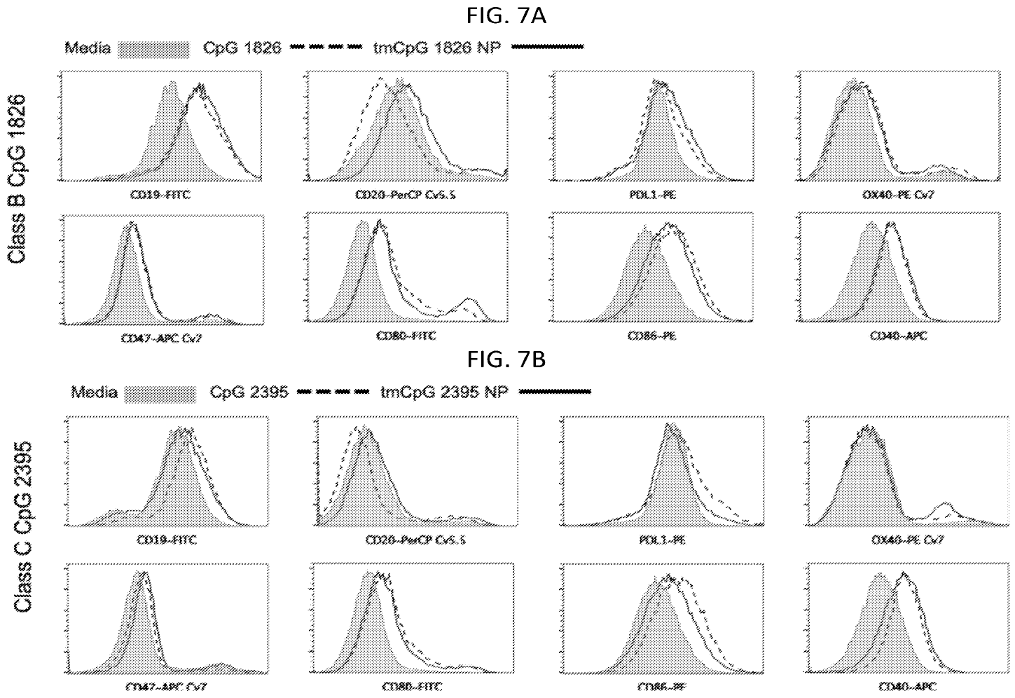

Here, targetable surface marker changes on A20 cells were evaluated when treated with class B (FIG. 7A) or class C (FIG. 7B) tmCpG NPs to inform future rational combination therapies. For B cell markers, CD19 was evaluated as it is a target for a number of antibody and cellular therapies. Treatment with either class B or class C CpG and tmCpG NPs increased CD19 expression in A20 cells compared with media controls (P<0.01) as seen in increased mean fluorescent intensity. Conversely, CD20 is a target for antibody therapies including rituximab or ibritumomab-tiuxetan. CD20 expression was higher in class B tmCpG 1826 NP treated lymphoma cells compared with free CpG 1826. Similarly, CD20 expression was also higher in class C tmCpG 2395 NP treated lymphoma cells compared with free CpG 1826. Of note, treatment with free class C CpG 2935 caused a reduction of CD20.

Lymphomas that overexpress programmed cell death ligand 1 (PDL1) demonstrate clinical responses to checkpoint inhibitors. A20 lymphoma cells normally overexpress PDL1. Treatment with either class of CpGs in free or nanoparticle form did not change PDL1 expression on A20 cells. For OX40 expression, treatment with class B tmCpG 1826 NPs (9.14%) resulted in similar expression levels compared with CpG 1826 (10.31%) and media (6.59%). By contrast, class C tmCpG 2395 NPs (14.88%) and class C CpG 2395 (12.88%) caused a significant increase in the number of cells expressing OX40 compared with media (7.4%).

Class B tmCpG 1826 NP (35.75%) and CpG 1826 (32.58%) led to an increase in number of CD47 positive cells compared with media control (14.55%). Class C tmCpG 2395 NP (17.85%) led to significantly higher numbers of CD47 positive cells compared with CpG 2395 (13.33%) or media control (12.69%). Free and tmCpG NP treatment groups for both class B and class C CpG sequences led to increased expression of B cell maturation markers including CD80, CD86, and CD40 compared with media controls. Differences in expression of these markers were small between free and tmCpG NP treatment groups.

Local Treatment with tmCpG NPs Reduced Treated Tumor Growth

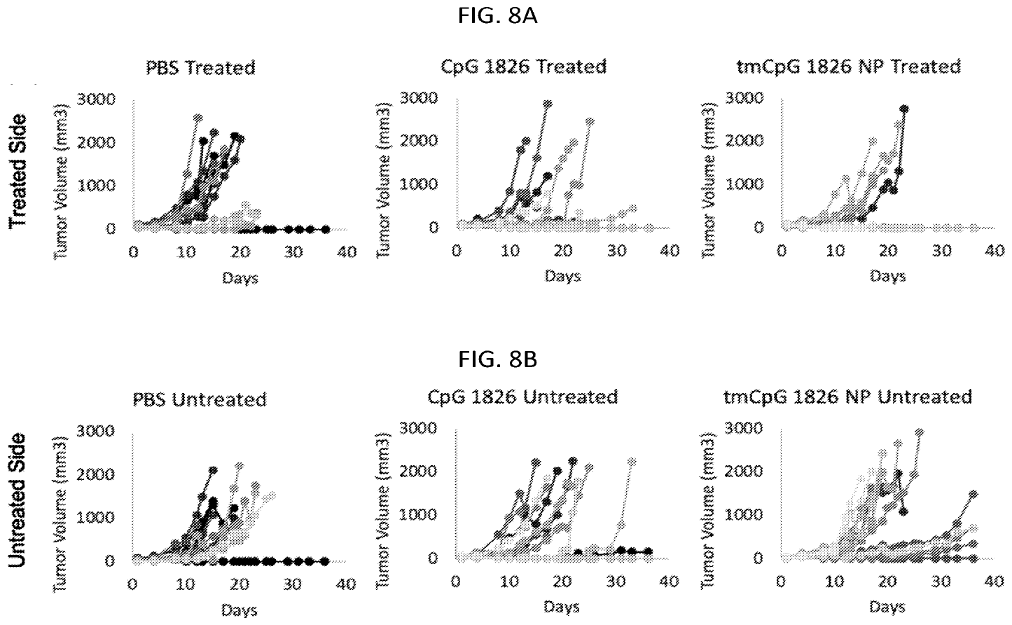

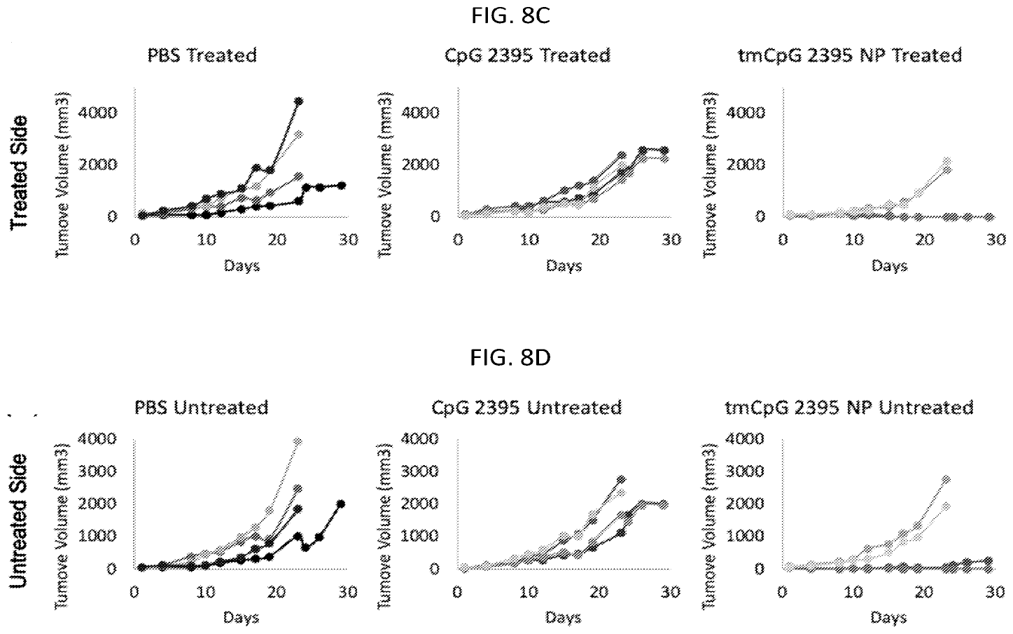

Lymphoma A20 cells were implanted on both left and right flanks of BALB/c mice. The larger tumor was treated with PBS, free CpG, or tmCpG NPs intra-tumorally (IT) on days 1, 4, and 8. For class B CpG 1826 (n=15), the tmCpG NP treatment group had a significant (P=0.04) survival benefit compared with PBS treatment when a survival event was defined by total tumor volume >2 cm₃. The hazard ratio was 0.423 (95% CI [0.187-0.96]). Free CpG 1826 compared with PBS treatment led to improvement in survival (P=0.067). When a survival event was defined as either side tumor reached 2 cm³, both the free CpG and the tmCpG NP treatment groups had improved survival compared with PBS. However, 87% (13/15) of the PBS treated group had the treated tumor reach 2 cm³ while only 53% (8/15) of the free CpG group and 13% (2/15) of the tmCpG NP group had the treated tumor reach 2 cm³ (FIG. 8A-8B), suggesting an enhanced local effect of the tmCpG NP group. In addition, 60% (9/15) mice in the NP group had no detectable tumor on the treated side, while only 26.7% (4/15) mice in the free CpG group had no detectable tumors (P=0.07, chi-squared).

By comparison, when treated with class C CpG 2935 (n=4 per group), the tmCpG NP treatment group did not have a significant survival benefit compared with PBS or free CpG treatment. However, the hazard ratio was 0.305 between the tmCpG 2395 NP group vs. PBS control group and 0.255 between the tmCpG 2395 NP vs. free CpG 2395 group. One out of four mice in the NP group had complete resolution of both tumors till end of study (day 60), while all of the PBS and free CpG mice died before day 28. Fifty percent (2/4) of the tmCpG 2395 NP treated tumors had complete resolution of the tumor until euthanasia, while none of the PBS or free CpG 2395 treated tumors had a complete response on the treated side. (FIG. 8C-8D).

### Combination of Both Classes of CpGs in a Nanoparticle Form Provided the Strongest Abscopal Effect

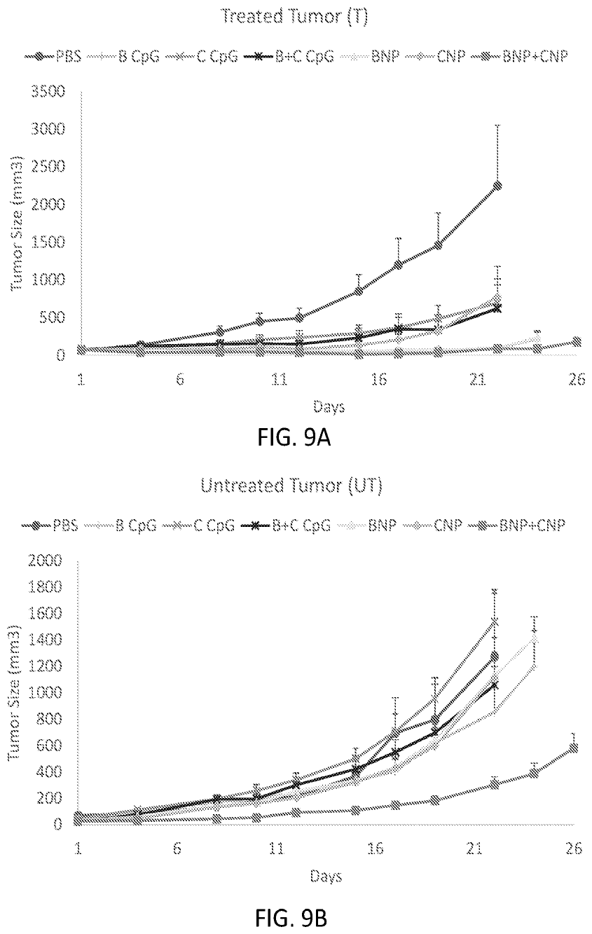

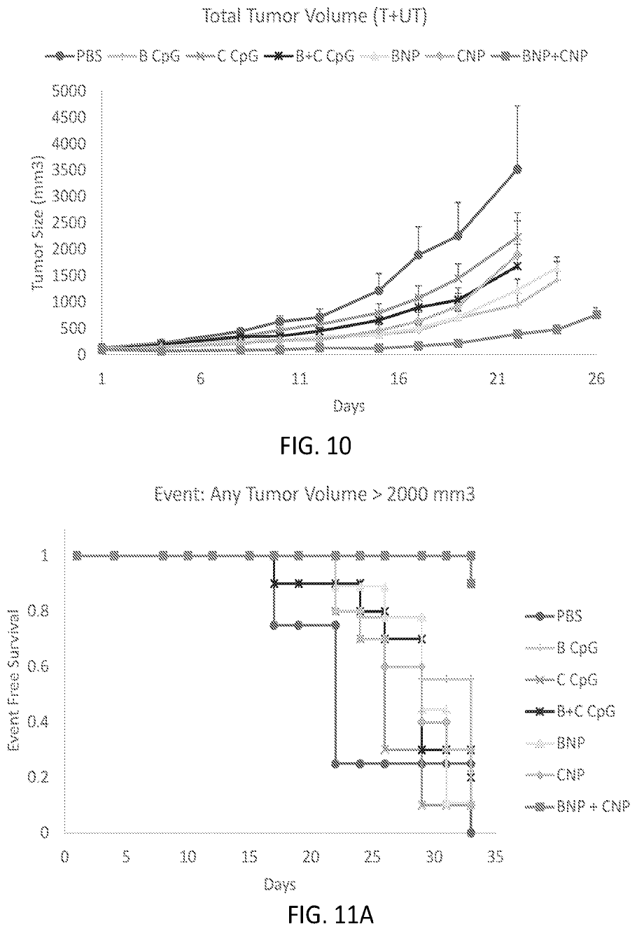

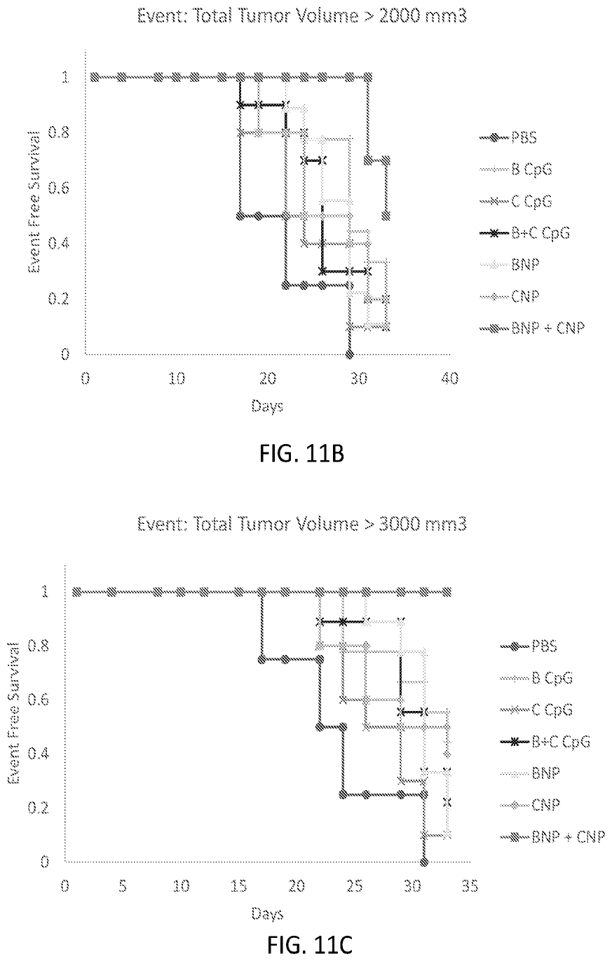

The combination of both classes of CpGs in a nanoparticle form provided the strongest abscopal effect. Treatment with B CpG or BNP or BNP+CNP had better direct tumor suppression compared with C CpG or CNP or B+C CpG (FIG. 9A). BNP+CNP treatment group had significantly better abscopal effect compared with all other conditions which are not different than PBS treatment group (FIG. 9B). The BNP+CNP group had significantly lower tumor volumes compared with all other conditions (FIG. 10). BNP+CNP had improved event free survival in comparison with all other groups for any tumor volume (FIG. 11A-11C).

At the end of the study at day 60 for this aggressive lymphoma, 2 out of 70 mice had no visible or palpable tumors. Both mice were part of the combination nanoparticle group and they would be considered as cured. No significant weight loss was seen in any of the groups as all mice increased weight. The slightly lower average of weight in the combination NP group was due to less tumor burden.

Moreover, the benefits of the administered NPs were observed with low CpG dosing. The present study used in these studies were much lower than previous studies where 50 ug of CpG per injection were typically used. In contrast, only 2.5 ug of CpG were used in the present study, i.e, 50 ul of 50 ug/ml of free CpG or 100 nM of CpG NPs. Not only was the therapeutic benefit realized at these low doses, but these low dose will result in less toxicity.

### Discussion

CpG therapy in B cell lymphomas is unique and clinically relevant because it not only can cause an anti-cancer immune response similar to melanoma, it also has direct cytotoxic effects. Here, it was demonstrated that the tmCpG NP platform is stable and able to deliver both class B and class C CpGs. Although changing the CpG sequence did not alter the self-assembled monolayer formation on the nanoparticles, class B CpGs were more stable during centrifugation and purification, while class C tmCpG NPs, due to sequence related issues, tended to aggregate more readily, and required additional steps to stabilize the construct. This was likely secondary to heating of the pellet during the high centrifugation process (CpG 2395 melting temperature is 66.5° C.), leading to duplex formation between two different nanoparticles. Controlling the temperature of the centrifugation steps prevented class C tmCpG NPs aggregate formation. Collectively, the results demonstrate the design of stable and robust class B and class C tmCpGNPs. This suggests that synthesis of this therapeutic platform can be practically scaled up in larger volumes.

The tmCpG NP design significantly improved CpG mediated cytotoxicity (apoptosis) against lymphoma cells compared with free CpGs even at relatively low concentrations (2.5 μg/ml). The tmCpG 1826 NPs reduced cell viability by 90%. The tmCpG NP platform enhances potency with reduced concentration of CpG required to elicit anti-tumor effect and therefore has the potential to minimize side effects of CpG therapy.

In addition, CpG induced apoptosis of lymphoma cells, especially in CLL, overcomes the initial increased proliferation. Here, it is also demonstrated that for difficult to treat lymphomas such as high-grade B cell lymphomas, TP53 mutated or deficient MCLs, the tmCpG NP platform is able to induce significant cell death by 5 days of treatment for both class B and class C CpG sequences. Class B CpGs tend to have stronger cytotoxic effects compared to class C CpGs, but both class B and class C CpG effect lymphoma cells.

Treatment with either class B or class C tmCpG NPs resulted in increased expression of CD19. As a result the NPs described herein may be used in combination with radiation, radioimmunotherapies, or immunotherapies, such as a CD19 targeting chimeric antigen receptor T cells (CAR-T). CAR-T therapy can be limited by problems with in vivo expansion of the engineered T cells and their durability. CpGs not only promote B cell and dendritic cell maturation and growth, but also co-stimulate T cells. In combination with tmCpG NPs, better tumor binding and expansion of CAR-T cells may be achieved.

CpG stimulation led to variable changes in CD20 expression. CpG treatment led to increased CD20 expression for marginal zone lymphomas and CLLs, or reduced levels in follicular lymphomas and MCL. Both class B and class C tmCpGNPs increased CD20 expression while free CpGs either did not alter or reduced expression of CD20. This suggests that the nanoparticle CpG platform would be most ideal as a lymphoma therapeutic as one would expect synergy with anti-CD20 therapies such as rituximab.

CD47, the “don't eat me” signal, is often over-expressed on lymphoma cells to evade macrophage-mediated phagocytosis. Increase in CD47 percentage on A20 cells after treatment with tmCpG NPs, though only by 5%, could further impede macrophage mediated anti-lymphoma immunity. However, A20 cells, as a CD47 expressing cell line, did respond to anti-CD47 antibody therapy. Anti-CD47 antibody (Hu5F9-G4) combined with rituximab showed responses in both DLBCL and follicular lymphoma patients.

Similarly, the PD-1 pathway is used by cancer cells to evade the immune system. A20 cells naturally have high expression of PD-L1 and tmCpG NPs did not further increase PD-L1 expression. In CT26 mouse models, intratumor injections of CpG combined with anti-PD1 therapy, showed rapid T cell infiltration and generation of multifunctional CD8+ T cells. This supports the use of the NPs described herein with immunotherapies.

Although a significant difference in overall survival between free CpG treated mice and tmCpG NP treated mice was not observed, class B tmCpGNPs had stronger direct cytotoxic effects at the treated tumor site with most of the mice euthanized because of larger untreated tumors. The overall tumor burden of the tmCG NP treated group was lower than the free CpG group. One out of four mice treated with class C tmCpG 2395 NPs had a complete response of both the treated and untreated tumor.

Overall, the results demonstrate that NPs formulated to deliver class B and class C CpGs treat lymphoma. This platform significantly improved the cytotoxic efficacy of CpGs against lymphoma cells. Though effective, class C tmCpGNP designs can be further optimized by forming CpG duplexes directly on the AuNP or prior to nanoparticle conjugation.

### Materials and Methods

### Particle Synthesis

Citrate stabilized AuNPs that are 15 nm, 30 nm, and 50 nm in diameter were purchased from Ted Pella, or synthesized using the Turkevich method.₂₇ Modified CpG designs were purchased from Integrated DNA Technology (IDT). All tmCpGs were uncapped by incubation with 100 mM dithiothreitol (Sigma-Aldrich) in sodium phosphate solution, pH 8.5 (Boston BioProducts), and eluted though Illustra NAP-5 columns (GE Healthcare) with sodium phosphate solution, pH 6.5, after 1 h incubation. Uncapped CpG sequences were added to citrate stabilized gold nanoparticles; then the solution is brought to 1×PBS with 0.1% Tween20 (Bio-Rad). The particles were collected and washed with PBS through centrifugation (×3). 15 nm, 30 nm, 50 nm particles were spun at 14,000×g, 7000×g, and 5000×g, respectively for 20 min.₁₅

### Viability Assay

Cell viability was quantified using Promega's CellTiter assay. Cells were plated at a density of 104 live cells per well. Media were used to dilute the free CpG or tmCpG NPs and filtered through a 0.22 μm syringe filter (Celltreat). The plate was cultured for 72-60 h prior to viability measurement. CellTiter reagent (Promega) was added to each well and absorbance was read at 490 nm using BioTek plate reader after 90 min of incubation.

### Apoptosis Assay

After 24 h of treatment of media, free CpG, or tmCpG NPs, the cells were collected, washed, and resuspended in staining buffer. The cells were stained with annexin V-FITC and propidium iodide (PI) per apoptosis kit instructions (BD Biosciences). The total apoptotic cells were analyzed as the total annexin V-positive cell population, in both the PI positive and negative gates (annexin V-FITC+/PI−) and (annexin VFITC+/PI+) cells.

### Surface Marker Assay

After 72 h of treatment of media, free CpG, or tmCpG NPs, the A20 cells were collected, washed, stained with LIVE/DEAD (Invitogen), and a master mix of either anti-CD19 (FITC), CD20 (PerCP-Cy5.5), PDL1 (PE), OX40 (PE-Cy7), CD47 (APC-Cy7), or anti-CD80 (FITC), CD86 (PE), CD40 (APC). All antibodies were purchased from Biolegend.

### Cytokine ELISAs

Supernatant of A20 cells treated with media control, class B or class C free CpG, or tmCpG NPs for 72 h or JAWSII cells for 24 h was collected for cytokine analysis. The IL-6 and TNFa levels were measured following standard protocol of the OptEIA Mouse IL-6 ELISA Set and TNF ELISA Set (BD Biosciences).

### Dual Tumor Lymphoma Model

All animal work was conducted in accordance with Northwestern University's IACUC under approved animal protocol (ISI00002415). 10₆ A20 cells in 200 μl of PBS were injected subcutaneously in bilateral flanks of BALB/c mice (Jackson Laboratories). For the class B experiment, 50 μl of either PBS, CpG 1826 (50 μg/ml), and tmCpG 1826 NP (100 nM) was injected intratumorally into the larger tumor on days 1, day 4, and day 8 (n=15). The length and width of tumors were subsequently measured 3 to 4 times a week.

### Statistics

All in vitro experiment statistics were analyzed using t tests. Significance was assigned at the α=0.05 level. All in vivo experiments were analyzed by the Quantitative Data Science Core, RHLCCC. Differences in survival were assessed using the log-rank test. Hazard ratios were calculated using the Cox proportional hazard model.

### Citrate Stabilized Gold Nanoparticles Synthesis Method

50 ml of 0.25 mM gold salt was heated to boil. Then, 170 ul of a 340 mM sodium citrate aqueous solution was rapidly added. Stirring and heating were continued for 1 hr.

### Magnetic Hollow Gold Nanoshell Synthesis

Silver core synthesis. 50 ml of 0.2 mM silver nitrate (AgNO₃) and 0.5 mM sodium citrate were heated on a stir-plate for 30 min. 1 ml of 100 mM sodium borohydride was added to the solution for 2 hr to form silver seeds. The silver seeds were then cooled to room temperature prior to the addition of 1 ml of 200 mM hydroxylamine. The solution was stirred for 5 min and 100 μl of 0.1 M AgNO₃ was added to the solution to form silver cores overnight (>18 hrs).

Synthesis of magHGN. For every 10 ml of silver cores, 1 ml of 5.5% Tween-20 was added to the solution prior to adding 75 μl of 5 mM 3-mercaptopropyltrimethoxysilane (MPTMS). The solution was stirred for 4 hr and the cores were collected using 10,000 Da molecular weight cutoff centrifuge filters. The MPTPS-coated Ag cores were suspended in a 0.1% Tween 20 solution and 300 μl of washed IONPs was added. The MPTMS-Ag and IONP solution was incubated at 50° C. for 18-20 hr. The particles were then washed with MQ water through centrifugation to remove excessive IONPs. 200 μl of 200 mM hydroxylamine was added 5 min prior to adding 10 μl of 0.1 M AgNO₃ to form the second layer of silver. The solution was stirred for three days to form Ag-IO-Ag complexes. 25 mM Au salt (HAuCl₄) was added to the complexes to form magHGNs.

Iron oxide washing. 2 ml of IONP (1.5×10¹⁶ particle/ml) in toluene was diluted with ethanol to 10 ml. The particles were pulled down using a 1 T magnet and washed with 1 M tetramethylammonium hydroxide (TMAOH) three times and with ethanol for another two times. The IONPs were resuspended in 2 ml 0.1% water prior to addition to MTPMS-Ag cores. Particles were sonicated in between every washing step.

PEG conjugation of magHGN. Freshly synthesized magHGNs were washed with MQ water through three centrifugation steps to remove excess reagents. The magHGNs were sonicated and resuspended in MQ water to the original volume. 100 μl of 10 mM methyl-polyethylene glycol-thiol (mPEG-SH) of 5,000 MW were added to every 10 ml of magHGNs. The solution was mixed for 24 hr at room temperature. Then, the salt concentration of the solution was brought to 1×PBS with 0.1% Tween 20 and incubated for another 24-48 hr. PEGylated magHGNs were washed with PBS through three centrifugation steps to remove excess PEG.

### CpG Conjugation on Gold Nanoparticles

Modified CpG ODN designs were purchased from Integrated DNA Technology (IDT). Uncapped CpG sequences (0.5 μM end concentration) were added to citrate-stabilized gold nanoparticles or hollow gold nanoshells for 24 hrs. The solution was brought to 1× phosphate buffered saline (PBS) and 0.1% Tween 20 and placed on a nutator for another 24 hrs. The particles were then collected and washed with PBS through three centrifugation steps.

### Gold Nanovaccine Synthesis

Carboxyl-PEG-thiols were added to 30-nm gold colloid solution (2×10¹¹ particles/ml) with an end concentration of 5 μM and incubated for 24 hrs. The solution raised to 0.1 M NaCl, 10 mM sodium phosphate, and 0.1% Tween 20. The excessive PEG molecules were removed from the AuNP solution by three centrifugation-washing steps at 7,000 g for 20 mins with PBS. After the carboxyl-PEG-AuNPs were suspended in MES buffer, 5 μl of 44 mM EDC and 5 μl of 59 mM sulfo-NHS linkers were added per ml of particle-MES solution and incubated for 15 mins at room temperature. The peptides (50 μg) were then added to the particles per ml of solution, and the mixture was incubated for one hour. Hydroxylamine (10 mM) was added to quench any unbound EDC/NHS for an additional hour. The peptide-coated particles were then centrifuged and washed three times with PBS. After the final PBS wash/centrifuge cycle, the supernatant was removed, and the particle pellet was re-suspended in 200 μl of PBS. The sample was sonicated and stored in the refrigerator until used.

### Mixed Class B and Class C Experiments

A total of 70 BALB/c mice were used in a dual tumor model with A20 cells. 4 mice for PBS and 9-10 mice per each other condition: free class B CpG (B CpG), free class C CpG (C CpG), free classes B and C CpGs (B+C CpG), class B tmCpG NPs (BNP), class C tmCpG NPs (CNP), and combined class B and C tmCpG NPs (BNP+CNP). When the tumors were 5-7 mm in diameter, the larger tumor was injected on days 1, 4, and 8. These were all female mice. The injections were 50 uls intratumorally on days 1, 4, 8. The concentration for CpGs was at 50 ug/ml and the nanoparticles were at 100 nM (or 50 ug/ml of CpG DNA). Mice that did not have measurable tumor on that day were excluded from the study. They all eventually grew tumors and were euthanized according to IACUC protocols when the tumors were too large.

1. A composition comprising a class B CpG conjugated nanoparticle and a class C CpG conjugated nanoparticle, wherein the class B CpG conjugated nanoparticle comprises a first nanoparticle core and a class B CpG conjugated thereto and wherein the class C CpG conjugated nanoparticle comprises a second nanoparticle core and a class C CpG conjugated thereto.

2. The composition of claim 1, wherein the class B CpG conjugated nanoparticle comprises a first spacer interposed between the class B CpG and the first nanoparticle core and/or the class C CpG conjugated nanoparticle comprises a second spacer interposed between the class C CpG and the second nanoparticle core.

3. The composition of claim 2, wherein the first spacer and/or the second spacer comprises an oligoethylene glycol and/or an oligonucleotide.

4. The composition of claim 1, wherein the first nanoparticle core and/or the second nanoparticle core comprises gold.

5. The composition of claim 4, wherein the class B CpG conjugated nanoparticle comprises a first gold nanoparticle core, a class B CpG, and a first spacer, the first spacer comprising the oligo ethylene glycol and a polyT oligonucleotide, and wherein the class C CpG conjugated nanoparticle comprises a second gold nanoparticle core, a class C CpG, and a second spacer, the second spacer comprising the oligoethylene glycol and a polyT oligonucleotide.

6. A pharmaceutical composition comprising the composition according to claim 1 and a pharmaceutically acceptable excipient, carrier, or diluent.

7. The pharmaceutical composition of claim 6, wherein the pharmaceutical composition is an injectable formulation.

8. A method for the inhibition of growth, proliferation, or killing of a cell, the method comprising contacting the cell with the class B CpG conjugated nanoparticle and/or the class C CpG conjugated nanoparticle according to claim 1 under conditions sufficient for inhibiting the growth, proliferation, or killing of the cell.

9. The method of claim 8, wherein contacting the cell with the composition leads to apoptosis.

10. A method for the increased expression of B cell markers, immune modulatory markers, and maturation markers, the method comprising contacting a cell with the class B CpG conjugated nanoparticle and/or the class C CpG conjugated nanoparticle according to claim 1 under conditions sufficient for increasing expression of a B cell marker, an immune modulatory marker, or a maturation marker of the cell.

11. The method of claim 10, wherein expression of CD19, CD20, CD40, CD47, CD80, CD86, OX40, or any combination thereof is increased.

12. A method for the increased expression of cytokines, the method comprising contacting a cell with the class B CpG conjugated nanoparticle and/or the class C CpG conjugated nanoparticle according to claim 1 under conditions sufficient for increasing expression of a cytokine of the cell.

13. The method of claim 12, wherein expression of IL-6, TNF, or a combination there of is increased.

14. The method of claim 8, wherein the cell is a hematologic cancer cell.

15. The method of claim 14, wherein the cell is a lymphoma cell.

16. The method of claim 12, wherein the cell is an immune cell.

17. The method of claim 16, wherein the cell is an antigen presenting cell or a lymphocyte.

18. (canceled)

19. A method for the treatment of a subject in need of a treatment for a cancer, the method comprising administering a therapeutically effective amount of the class B CpG conjugated nanoparticle and/or the class C CpG conjugated nanoparticle according to claim 1 or a pharmaceutical composition thereof, the pharmaceutically composition further comprising a pharmaceutically acceptable excipient, carrier, or diluent.

20. The method of claim 19, wherein the cancer is a hematological cancer.

21. The method of claim 20, wherein the cancer is a lymphoma.

22. The method of claim 19 further comprising co-administering an immunomodulatory therapy.

23. (canceled)
